(19) United States
(12) Patent Application Publication
(10) Pub. No.: US 2016/0279436 A1
(43) Pub. Date: Sep. 29, 2016

(54) Phototherapy Apparatus with Dosage Control

(71) Applicants: Sean Xiaolu Wang, Wilmington, DE (US); Luis De Taboada, Carlsbad, CA (US); Brian Pryor, Newark, DE (US)

(72) Inventors: Sean Xiaolu Wang, Wilmington, DE (US); Luis De Taboada, Carlsbad, CA (US); Brian Pryor, Newark, DE (US)

(73) Assignee: BWT PROPERTY, INC., Newark, DE (US)

(21) Appl. No.: 14/665,251

(22) Filed: Mar. 23, 2015

(51) Int. Cl. A61N 5/06

(52) U.S. Cl. CPC A61N 5/06; A61N 2005/067

(57) ABSTRACT

A phototherapy apparatus with precise dosage control is disclosed. The phototherapy apparatus incorporates means for tracking any combination of the following: position, direction of motion, velocity, or acceleration of the therapeutic light beam over the treatment area. The delivered light dosage is calculated based on these parameters and the intensity of the laser beam.

### FIELD OF THE INVENTION

This invention generally relates to a phototherapy apparatus, and more specifically to a phototherapy apparatus with precise dosage control for the delivery of a clinically safe and effective dose to target tissues.

### BACKGROUND

Phototherapy is a medical and veterinary technique which uses laser, light emitting diode (LED) or other types of light sources to restore, stimulate or inhibit cellular function, and prevent cell death. Recently, this technique has been widely used for treating soft tissue injury, chronic pain, and promoting wound healing for both human and animal targets.

Typically, the phototherapy procedure involves radiating light energy in the ultraviolet (UV), visible, near infrared, or infrared wavelength onto or into the patient's skin. It is highly desirable to precisely control the dose of light energy that is applied on a specific treatment area to achieve a safe and effective therapeutic effect. However, none of the existing phototherapy apparatus could provide this feature due to the following reasons. First, the therapeutic light generally has a non-uniform beam profile, e.g., the light intensity varies significantly from the center to the edge of the light beam. Thus the treatment area inevitably receives uneven dosages. Second, some therapeutic light (e. g. near infrared light) is invisible to the human eyes. In these cases, an aiming beam in the visible wavelength is generally provided to guide the therapy, i.e., to provide the user with a location for the invisible therapeutic light. However, due to their being generated by different light sources in wavelength and output power, the aiming beam generally has an intensity profile different from that of the therapeutic light, which prevents it from providing precise dosage guidance to the clinician or practitioner. Third, the practitioner or clinician usually needs to scan the therapeutic light beam to cover a large treatment area, making it even harder to track the exact dose delivered to any specific region of the area.

There thus exists a need for an improved phototherapy apparatus, which can provide real time monitoring of the delivered light dosage on the subject surface of the biological tissue for assisting the practitioner or clinician in precisely controlling the phototherapy procedure.

### SUMMARY OF THE INVENTION

It is the overall goal of the present invention to solve the above mentioned problems and limitations, and provide a phototherapy apparatus with precise dosage control. The phototherapy apparatus incorporates means for tracking any combination of the following: position, direction of motion, velocity, or acceleration of the therapeutic light beam over the treatment area. The delivered light dosage is calculated based on these parameters and the intensity of the laser beam.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates one exemplary embodiment of the phototherapy apparatus with dosage control.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to a phototherapy apparatus with precise dosage control. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

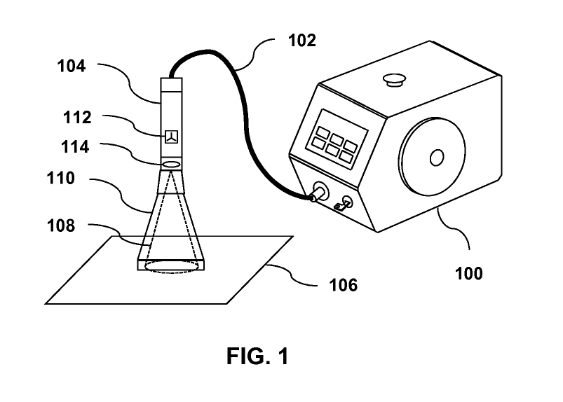

FIG. 1 illustrates one exemplary embodiment of the phototherapy apparatus with dosage control. Here the light source module 100 of the phototherapy apparatus comprises a high power diode laser operating at a near infrared wavelength of 980 nm. The output power of the diode laser is adjustable in the range of 0.5-15 watts for producing photochemical reaction, e.g. up-regulation and down-regulation of adenosine triphosphate (ATP), reactive oxygen species, and nitric oxide in the subject biological tissue 106. Although not exclusively, the photochemical reaction in turn produces one or any combination of the following therapeutic effects: (i) stimulating white blood cell activity; (ii) accelerating macrophage activity, growth factor secretion and collagen synthesis; (iii) promoting revascularization and micro-circulation; (iv) increasing fibroblast numbers and collagen production; (v) accelerating epithelial cell regeneration and speeding up wound healing; (vi) increasing growth-phase-specific DNA synthesis; (vii) stimulating higher activity in cell proliferation and differentiation; (viii) increasing the intra and inter-molecular hydrogen bonding. All these therapeutic effects combine to benefit the subject biological tissue 106.

Referring to FIG. 1, the phototherapy apparatus comprises an optical fiber 102 and an output wand 104 for delivering the laser light from the light source module 100 onto the surface of the subject biological tissue 106. The laser light 108 is absorbed by the chromophores (e.g. cytochrome c oxidase) of the biological tissue to trigger the above disclosed photochemical reactions. A spacer 110 is employed to control the distance from the output port of the wand 104 to the surface of the biological tissue 106. The divergence angle of the laser beam 108 is set by the numerical aperture of the optical fiber 102. Preferably, an optical lens 114 is mounted at the output port of the wand 104 to provide more precise control of the divergence angle of the laser beam 108. Thus the intensity of the laser beam on the surface of the biological tissue 106 is set by the power of the laser 100 and the length of the spacer 110. In the represented embodiment of the invention, the output wand 104 further comprises an embedded accelerometer 112, which is used as a tracking element for tracking any combination of the following: position, direction of motion, velocity, or acceleration of the wand 104 (hence the laser beam 108) at any point in time over the surface of the biological tissue 106. With these parameters, the scanned surface area and time of duration of the laser beam are determined and recorded with a processor unit (not shown). The delivered light dosage (which is a product of laser intensity and time of duration) on any specific location within the scanned area of the tissue surface is then calculated by multiplying the laser intensity with the time of duration of the laser beam over that location. In this manner, the practitioner or clinician can precisely control the delivered light dosage.

The disclosed phototherapy apparatus can be used in other fields as well, such as photo-dynamic therapy, where the light source is used to activate a photosensitizing drug, or in aesthetic treatments such as acne treatment, wrinkle removal, skin-tightening, etc.

In the foregoing specification, specific embodiments of the present invention have been described. However, one of ordinary skill in the art appreciates that various modifications and changes can be made without departing from the scope of the present invention as set forth in the claims below. The numerical values cited in the specific embodiment are illustrative rather than limiting. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. A phototherapy apparatus for treating biological tissue, said phototherapy apparatus comprising:
at least one light source for producing therapeutic light at a predetermined output power;
an output wand for delivering the therapeutic light onto a surface of the biological tissue through an output port;
an tracking element for tracking any combination of the following: position, direction of motion, velocity, or acceleration of the therapeutic light over the surface of the biological tissue; and
a processor unit for determining a delivered light dosage onto the surface of the biological tissue based on any combination of the following: position, direction of motion, velocity, or acceleration of the therapeutic light from the tracking element.

2. The phototherapy apparatus of claim 1, wherein the at least one light source comprises a near infrared laser.

3. The phototherapy apparatus of claim 1, wherein the tracking element is an accelerometer.

4. The phototherapy apparatus of claim 1, wherein the tracking element is embedded in the output wand.

5. The phototherapy apparatus of claim 1, further comprising a spacer element for controlling a distance from the output port of the wand to the surface of the biological tissue.

6. The phototherapy apparatus of claim 1, further comprising an optical lens at the output port of the output wand for controlling a divergence angle of the therapeutic light.
